(19) United States
(12) Patent Application Publication
(10) Pub. No.: US 2014/0100236 A1
(43) Pub. Date: Apr. 10, 2014

(54) Use of selectively moisture-adjusted tabletting material in the production of mechanically stable tablets which contain at least one hydrate-forming active substance and/or adjuvant relevant to the mechanical stability of the tablets, particularly arginine-containing tablets

(71) Applicants: Maria BUSL, Gau-Algesheim (DE); Eduard BALTHES, Ingelheim am Rhein (DE)

(72) Inventors: Maria BUSL, Gau-Algesheim (DE); Eduard BALTHES, Ingelheim am Rhein (DE)

(73) Assignee: BOEHRINGER INGELHEIM INTERNATIONAL GMBH, Ingelheim am Rhein (DE)

(21) Appl. No.: 14/048,445

(22) Filed: Oct. 8, 2013

(30) Foreign Application Priority Data
Oct. 9, 2012 (EP) 12187796.3

(51) Int. Cl. A61K 47/18

(52) U.S. Cl. CPC A61K 47/183
USPC 514/263.21; 514/784; 514/635; 514/338

(57) ABSTRACT

The present invention relates inter alia to the use of selectively moisture-adjusted tabletting material in the preparation of mechanically stable oral tablets which contain at least one hydrate-forming active substance and/or adjuvant relevant to the mechanical stability of the tablets, particularly arginine-containing oral tablets.

### FIELD OF INVENTION

The present invention relates inter alia to the use of selective moisture-adjusted formulations or tabletting material in the production of mechanically stable oral tablets which contain at least one hydrate-forming active substance and/or adjuvant relevant to the mechanical stability of the tablets, particularly arginine-containing oral tablets, to such formulations, tabletting material or tablets which may be obtained by selective moisture adjustment (e.g. moisture conditioning or drying), as well as to methods for their preparation.

### BACKGROUND OF THE INVENTION

L-arginine-containing tablets may form cracks or become brittle under moist climatic conditions.

Methods of tablet production are described in the patent literature (cf. for example JP 2010254580, JP 2010-27011, US 2008/0145424, JP 2005-298373, WO 2009/121945), by means of which the problem of the brittleness of arginine-containing tablets (particularly those with a higher arginine content) can be circumvented. However, the possibilities are limited to coating the tablets with a moisture repellent coating, adding a hydrophobic additive or wet-granulating the starting materials. Particularly in the case of sensitive, particularly moisture-sensitive products, it may be the case that for a number of very different reasons wet granulation is out of the question, which means that processing in the form of a dry granulated material or a direct tabletting mixture is absolutely essential. According to our present knowledge such methods of processing L-arginine-containing tabletting material have not hitherto been described in the patent literature.

Moreover, to ensure the stability of moisture-sensitive products in many cases considerable expense is required, for example expensive packaging designs (e.g. blister systems consisting of high barrier films such as for example Aclar, aluminium or pouch systems, possibly with an additional desiccant or bottles containing desiccants) or complex processing steps (e.g. defined climatic conditions during packaging, a drying step included in the manufacture, subsequent drying, bulk drying of the tablets, climate-controlled storage of the bulk goods, short machinery downtimes). This gives rise to an obvious need to find new methods of ensuring the mechanical stability of tablets by specific processing of the tabletting material from the outset, thereby saving the expense of subsequent process steps.

### SUMMARY OF THE INVENTION

The present invention relates inter alia to the use of selective moisture-adjusted tabletting material in the production of mechanically stable oral tablets which contain at least one hydrate-forming active substance and/or adjuvant relevant to the mechanical stability of the tablets, particularly arginine-containing oral tablets. In particular, the present invention relates inter alia to the use of moisture-conditioned tabletting material or moisture-conditioned final mixtures (or alternatively their individual components or mixtures of the individual components) in tablet manufacture for the controlled selection of the mechanical properties (e.g. hardness) of tablets.

Also, the present invention relates in particular to the production of mechanically stable tablets from dry granulated material, dry compacted material or from powder mixtures, e.g. for direct tabletting, which contain at least one hydrate-forming active substance and/or adjuvant relevant to the mechanical stability of the tablets, particularly ones that contain the adjuvant L-arginine (or a hydrate-forming agent having the same or analogous characteristics). The production of mechanically stable tablets from wet granulated material which contain at least one hydrate-forming active substance and/or adjuvant relevant to the mechanical stability of the tablets, particularly those containing the adjuvant L-arginine (or a hydrate-forming agent with the same or analogous characteristics) is also described.

According to the invention this can be achieved by a selective moisture adjustment, i.e. selective moisture conditioning or drying of the tabletting material (e.g. of the final mixture, individual components or mixtures of components of the formulation), for example as a process step before the tabletting. In this way it is possible to further process corresponding starting material (particularly containing L-arginine) to produce tablets which have a durable mechanical stability and to ensure this over a very wide humidity range.

Some of the terms used herein may be described in more detail hereinafter:

Arginine: unless explicitly mentioned, this always refers to the proteinogenic amino acid L-arginine, which can be used as an adjuvant and/or active substance.

Mechanical stability: refers to a mechanically stable tablet core if possible with no cracks, breaks or broken edges on the score-line, with no broken-off tablet fragments or so called deckle-edges. The tablet core should if possible not break up or fragment when gripped or during normal handling. The term “mechanically stable” is not intended to contain any quantitative statement as to the breaking force.

Dry compacted material formulation: final mixture ready for tabletting, produced by dry compacting of the adjuvants and active substances, optionally with final addition of an external phase (e.g. magnesium stearate).

(Final) powder mixture/powder formulation: mixing of all the components in powder form without a granulation process.

Wet granulation/wet granulated material: adhesive or deposited granulated materials which are produced with the addition of liquid (e.g. water, water/ethanol mixtures, water-containing adhesive solutions of polymer binders such as gelatine or starch).

Dry granulated material: granulated material produced without the addition of granulating liquid, but under the effect of physical parameters such as pressure or temperature.

DVS measurement: measurement of the water absorption of a sample as a function of relative humidity and temperature using a highly sensitive sorption scale.

A hydrate-forming active substance and/or adjuvant is an active substance and/or adjuvant which may interact with water or moisture as follows, for example:

Hydration water/water of crystallization: water that is stoichiometrically bound in the crystal lattice of a substance.

Adsorption water: water that accumulates on solid surfaces.

Swelling water: water that is incorporated in polymer structures and leads to swelling by forcing the individual layers apart.

Capillary water: water that is incorporated in capillary-porous structures.

Accordingly, formation of a hydrate according to this invention may be characterized, for example, by binding water (e.g. in a crystal lattice such as crystallization water), adsorbing water (e.g. on a solid surface such as adsorption water), or incorporating water (e.g. in capillary-porous structures or in polymer structures, such as capillary water or swelling water).

In an embodiment, a hydrate-forming active substance and/or adjuvant may be a moisture sensitive active substance and/or adjuvant, which may bind water (e.g. in a crystal lattice, e.g. as crystallization water), adsorb water (e.g. on a solid surface, e.g. as adsorption water), or incorporate water (e.g. in capillary-porous structures or in polymer structures, e.g. as capillary water or swelling water).

r.h. denotes relative humidity.

Unconditioned material/tabletting material: material or tabletting material which has not been stored and conditioned under defined climatic conditions, with the aim of achieving a specific moisture content.

Phase transition in hydrate-forming active substances or adjuvants: the relative humidity at which the hydrate-forming agent water (particularly water of crystallization) is incorporated and changes from an anhydrate or a lower-valency form into a higher valency hydrate form, or vice versa. This relative humidity is characteristic of the respective hydrate-forming agent; if a hydrate-forming agent incorporates water in several steps, there are several phase transitions in this substance. For example: phase transition for pure L-arginine: 38% r.h./25° C. with incorporation of about 1.5 mol of water per 1 mol of L-arginine; in mixtures comprising L-arginine the phase transition is typically between 38 and 40% r.h./25° C., but in the test formulation described herein by way of example it starts, at latest, at 40% r.h./25° C.

Selective moisture conditioning: here the term is used particularly to mean the following: the hydrate-forming active substance or adjuvant is moisture-conditioned as an individual substance or within a formulation such that in the hydrate-forming agent the phase transition has just been exceeded, i.e. in the case of L-arginine roughly 1.5 mol of water of crystallization have been incorporated per 1 mol of L-arginine. If the water of crystallization is incorporated step by step, the selective conditioning must be linked to the climatic conditions applicable thereto.

Sorption cycle: the powder/granulated material/compacted material/product absorbs moisture as the result of an increasing supply of relative humidity in the air.

Desorption cycle: the powder/granulated material/compacted material/product releases moisture as a result of the decreasing supply of relative humidity in the air.

### BRIEF DESCRIPTION OF THE DRAWINGS

FIG. 1 shows the sorption curve of linagliptin+pioglitazone HCl compacted material at 25° C. (sorption and desorption cycle; change water content vs. rel. humidity). Tablets produced from tabletting material pre-conditioned at 25° C./45% r.h. were subsequently stored in very different humidity ranges (arrows; for discussion of the cases 1-4 see the description herein).

### DETAILED DESCRIPTION OF THE INVENTION

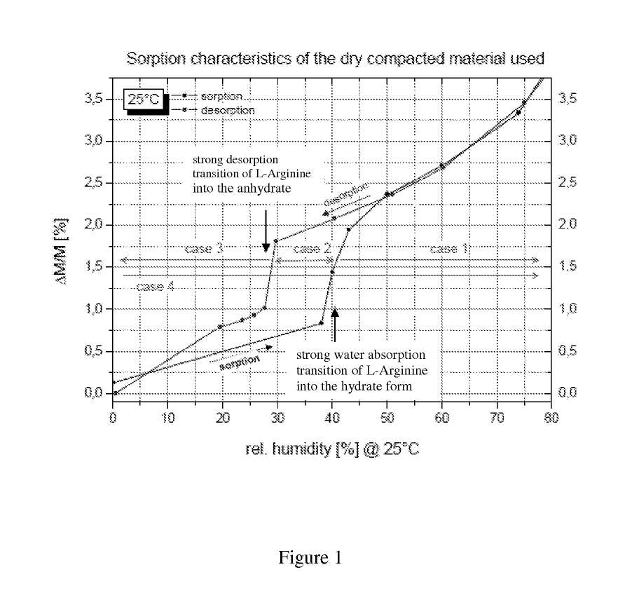

Within the scope of the present invention it has now been found that the hydration of L-arginine which occurs in the tablet under the effect of moisture from about 38-40% r.h./25° C. may be responsible for mechanical instability (see the Examples, black sorption curve in FIG. 1). As a result of the incorporation of water in the crystal lattice of L-arginine, the size dimensions of the crystal unit cell change and take up more space, which may eventually cause cracks or fractures in the tablet.

Within the scope of the present invention, the particular moisture range or water loading (m/m) based on L-arginine that is the minimum required to enable mechanically stabled tablets to be produced has also now been found.

As an inventive idea for solving the problem it is now proposed, for example, to condition the tabletting material (e.g. dry granulated material, dry compacted material or powder mixture) or the formulation (e.g. final mixture or alternatively the individual components or mixtures of the individual components thereof) before tabletting at least to a relative humidity which in the sorption cycle corresponds to the phase transition of the hydrate-forming agent present (example of “selective moisture conditioning”) or to use correspondingly moisture-conditioned material, if the uptake or incorporation of the water (particularly water of crystallisation) in the hydrate-forming agent and the change in the crystal lattice structure possibly associated therewith leads to brittleness or mechanical instability of the tablet.

If there are a plurality of hydrate-forming active substances and/or adjuvants in the formulation in which fracturing in the product can be prevented by selective moisture conditioning, the relative humidity to be selected in the sorption cycle for selective conditioning should be the one at which all the hydrate-forming agents that determine the mechanical stability have exceeded their phase transition. If a substance incorporates water (particularly water of crystallisation) step by step, during the conditioning it is essential to exceed the phase transition that is causally responsible for the fracturing/destruction of the product.

The moisture conditioning can be carried out by storage under defined climatic conditions and/or using a procedure as described herein.

It is thus possible to achieve optimization with regard to mechanical or physical stability or shelf life.

According to the invention, it is possible to ensure efficient and total hydration of one or more hydrate-forming active substances and/or adjuvants such as L-arginine by checking the amount of water taken up (e.g. by weighing) while at the same time circulating the tabletting material. For conditioning L-arginine-containing tabletting materials the provision of at least 38% r.h., preferably 40% r.h. at 25° C. is required in order to exceed the phase transition in the amino acid. In very moisture-sensitive formulations which contain one or more hydrate-forming agents, it is advisable to carry out the conditioning very close to the phase transition of the hydrate-forming agent which is last to exceed the phase transition crucial to the mechanical stability in the sorption cycle. In the case of L-arginine-containing moisture-sensitive formulations containing L-arginine as the only hydrate-forming agent, the mildest possible treatment is achieved with a setting of at least 38% r.h./25° C., preferably 40% r.h./25° C. Alternatively, for the formulation mentioned above, in principle any other temperature/humidity combination can be selected at which the same water content is obtained in the tablet as under the climate conditions mentioned above. For example, at 40° C. a relative humidity of about 47% r.h. would have to be selected to ensure the phase transition in L-arginine.

Accordingly, in one embodiment of the present invention, the moisture-conditioned L-arginine according to the invention refers to L-arginine which may be obtained by moisture conditioning at least 38% r.h., preferably 40% r.h. at 25° C. (e.g. 45% r.h./25° C.).

With the method proposed here according to the invention, the corresponding amount of water required to produce the hydrate form of all the hydrate-forming agents present for the mechanical stability of the formulation can be introduced into the tabletting material even before the tabletting process, by conditioning at defined climatic conditions, so that the restructuring of the particular crystal lattice takes place in the loose tabletting material. The result of this is that a change in the crystal lattice structure of the hydrate-forming substances in the tablet itself is avoided, thus ensuring the stability of tablets made from tabletting material pre-treated in this way. This makes it possible for the first time to obtain mechanically stable tablets from dry compacted material, dry granulated material or a powder mixture, which contain hydrate-forming adjuvants and/or active substances, using the manufacturing method described here. In the case of the L-arginine-containing dry compacted formulation it is possible for the first time to obtain a mechanically stable product by using the manufacturing method described here.

Within the scope of the present invention it is further proposed to use one or more selectively moisture-conditioned hydrate-forming active substances and/or adjuvants (e.g. L-arginine) in the relevant formulations in order to avoid adversely affecting the physical stability or shelf-life of the product by the uptake or incorporation of water (particularly water of crystallization) and a resulting structural change and/or increase in the volume of the hydrate-forming agent in the finished tablet. In this way the physical or mechanical stability or shelf-life is optimised.

It is further proposed within the scope of the present invention, in the manufacture of tablets which have hitherto contained one or more hydrate-forming active substances and/or adjuvants (such as L-arginine) in the formulation, which have been processed either dry or at relative humidity levels below the phase transition which is relevant to the stability of the tablets, to replace these with selectively moisture-conditioned corresponding active substances and/or adjuvants (e.g. L-arginine moisture-conditioned at at least 38 to 40% r.h./25° C.) in the formulation, particularly to prevent adverse effects on the physical stability or shelf life caused by the incorporation of water (particularly water of crystallization) in the hydrate-forming agents (e.g. during production, packaging or storage, particularly in the finished tablet) if the chemical stability of the product allows such a manufacturing process.

The intention of this is to keep the tablets physically stable, even at relative humidity levels above the phase transition of the hydrate-forming adjuvant and/or active substance relevant to the mechanical stability in the sorption cycle, e.g. to prevent the formation of cracks or fracturing, particularly as a result of the incorporation of water of crystallization with an associated change in the crystal lattice spacings.

The knowledge of the phase transitions during the release of water in the desorption cycle can also be used to ensure that after manufacture the product is not dried below a certain relative humidity that corresponds to the phase transition of the hydrate-forming adjuvant or active substance, if the release of water (particularly water of crystallization) in the hydrate-forming agent and the change in the crystal lattice structure possibly associated therewith leads to brittleness or mechanical instability of the tablet. If the formulation contains a number of hydrate-forming agents, during the drying of the product the phase transition of the particular hydrate-forming agent that releases its water first (particularly water of crystallization) and thus triggers fracturing is crucial to the mechanical stability.

Furthermore, the use of selectively moisture-conditioned or dried hydrate-forming active substances and/or adjuvants is intended to expand the relative humidity and temperature range of the production and/or storage of tablets, to simplify their manufacture, minimise the costs of suitable packaging and/or at the same time increase their shelf life.

Accordingly, the present invention provides a formulation (particularly a solid pharmaceutical formulation or composition, for example in the form of a tablet, blend or tabletting material) which contains a moisture-conditioned or dried hydrate-forming active substance and/or adjuvant (e.g. moisture-conditioned or dried L-arginine, particularly as adjuvant) as the active substance or adjuvant.

Furthermore, the present invention provides a formulation (particularly a solid pharmaceutical formulation or composition, for example in the form of a tablet, blend or tabletting material) which contains one or more active substances and/or adjuvants, one or more of said active substances and/or adjuvants being selectively moisture-conditioned or dried hydrate-forming active substances and/or adjuvants in the sense of the present invention (e.g. comprising in particular selectively moisture-conditioned or dried L-arginine, particularly as adjuvant).

Similarly, the present invention provides a formulation (particularly a solid pharmaceutical formulation or composition such as, for example, in the form of a tablet or tabletting material e.g. consisting of dry granulated material, dry compacted material or powder mixture) which contains a hydrate-forming active substance and/or adjuvant (e.g. L-arginine, particularly as adjuvant) which is obtainable or obtained by systematic moisture conditioning of the hydrate-forming active substance and/or adjuvant (e.g. L-arginine, particularly as adjuvant, e.g. on its own or in admixture, and/or in tabletted form), particularly by systematic moisture adjustment at least to the relative humidity which in the sorption cycle corresponds to the phase transition of the hydrate-forming active substance and/or adjuvant; in particular if the uptake or incorporation of the water (particularly water of crystallization) in the hydrate-forming agent and the change in the crystal lattice structure possibly associated therewith leads to brittleness or mechanical instability of the tablet.

Also, the present invention provides a formulation (particularly a solid pharmaceutical formulation or composition, e.g. in the form of a tablet or tabletting material, e.g. made up of wet granulated material) which contains a hydrate-forming active substance and/or adjuvant (e.g. L-arginine, particularly as adjuvant), which is obtainable or obtained by systematic drying of the hydrate-forming active substance and/or adjuvant (e.g. L-arginine, particularly as adjuvant, e.g. on its own or in admixture, and/or in tabletted form), particularly by controlled drying to a point not below the relative humidity which in the desorption cycle corresponds to the phase transition of the hydrate-forming active substance and/or adjuvant; in particular if the release of water (particularly water of crystallization) in the hydrate-forming agent and the change in the crystal lattice structure possibly associated therewith leads to brittleness or mechanical instability of the tablet.

Equally, the present invention provides a formulation (particularly a solid pharmaceutical formulation or composition such as for example in the form of a tablet or tabletting material, made up for example of dry granulated material, dry compacted material or powder mixture) which contains a hydrate-forming active substance and/or adjuvant (e.g. L-arginine, particularly as adjuvant), wherein the hydrate-forming active substance and/or adjuvant (e.g. L-arginine, particularly as adjuvant) is conditioned at least to the relative humidity of its phase transition relevant to the stability of the tablet in the sorption cycle.

Also, the present invention provides a formulation (particularly a solid pharmaceutical formulation or composition, such as for example in the form of a tablet or tabletting material made up for example of moist granulated material) which contains a hydrate-forming active substance and/or adjuvant (e.g. L-arginine, particularly as adjuvant), wherein the hydrate-forming active substance and/or adjuvant (e.g. L-arginine, particularly as adjuvant) is dried to a point that is not below the relative humidity of its phase transition relevant to the stability of the tablet in the desorption cycle.

Moreover, the present invention relates to a formulation (particularly a solid pharmaceutical formulation, mixture, final mixture, tabletting material or preparation, or composition, e.g. in the form of a tablet) comprising (or essentially consisting of) one or more selectively moisture-conditioned or dried hydrate-forming active substances and/or adjuvants (e.g. L-arginine, particularly as adjuvant), particularly those selectively moisture-conditioned or dried hydrate-forming active substances and/or adjuvants obtained by humidity adjustment (moisture conditioning or drying, e.g. of the individual components or mixtures of the individual components, the tabletting material, the final mixture or the formulation) which ensures that the hydrate-forming active substances and/or adjuvants are present in their hydrate forms that are relevant to the mechanical stability of the formulation, optionally together with one or more other active substances and/or other adjuvants.

Furthermore, the present invention relates to a formulation (particularly a solid pharmaceutical formulation, mixture, final mixture, tabletting material or preparation, or composition, e.g. in the form of a tablet) comprising (or essentially consisting of) one or more hydrate-forming active substances and/or adjuvants (e.g. L-arginine, particularly as adjuvant), optionally together with one or more other active substances and/or other adjuvants,

characterised in that the said one or more hydrate-forming active substances and/or adjuvants are selectively moisture-conditioned or dried,
particularly characterised in that the said one or more hydrate-forming active substances and/or adjuvants are selectively moisture-conditioned or dried hydrate-forming active substances and/or adjuvants of this kind, obtained by humidity adjustment (moisture-conditioning or drying, e.g. of the individual components or a mixture of the individual components, the tabletting material, the final mixture or the formulation) which ensures that the hydrate-forming active substances and/or adjuvants are present in their hydrate forms that are relevant to the mechanical stability of the formulation;
in particular if the uptake or incorporation of the water (particularly water of crystallisation) in the hydrate-forming agent and the optionally associated change in the crystal lattice structure leads to a brittleness or mechanical instability of the formulation (e.g. tablet), and/or
in particular if the release of the water (in particular water of crystallisation) in the hydrate-forming agent and the optionally associated change in the crystal lattice structure leads to mechanical instability of the formulation (e.g. tablet).

The present invention further relates to a formulation (particularly a solid pharmaceutical formulation or composition, e.g. in the form of a tablet or tabletting material consisting for example of dry granulated material, dry compacted material or powder mixture), obtained from or essentially consisting of:

one or more selectively moisture-conditioned hydrate-forming active substances and/or adjuvants (e.g. L-arginine, particularly as adjuvant), particularly those selectively moisture-conditioned hydrate-forming active substances and/or adjuvants obtained by moisture conditioning with a minimum conditioning humidity which ensures the phase transition of each hydrate-forming agent relevant to the mechanical stability of the tablet in the sorption cycle,
optionally one or more other active substances,
and optionally one or more other pharmaceutically acceptable adjuvants;
in particular if the uptake or incorporation of the water (particularly water of crystallisation) in the hydrate-forming agent and the change in the crystal lattice structure possibly associated therewith leads to a brittleness or mechanical instability of the tablet.

The present invention further relates to a formulation (particularly a solid pharmaceutical formulation or composition, e.g. in the form of a tablet or tabletting material consisting for example of dry granulated material, dry compacted material or powder mixture), obtained from or essentially consisting of:

one or more selectively moisture-conditioned hydrate-forming active substances, particularly obtained by moisture conditioning with a minimum conditioning humidity that ensures the phase transition of each hydrate-forming agent relevant to the mechanical stability of the tablet,
optionally one or more other active substances,
and optionally one or more adjuvants.

The present invention further relates to a formulation (particularly a solid pharmaceutical formulation or composition, e.g. in the form of a tablet or tabletting material consisting for example of dry granulated material, dry compacted material or powder mixture), obtained from or essentially consisting of:

one or more active substances,
one or more selectively moisture-conditioned hydrate-forming adjuvants, particularly obtained by moisture conditioning with a minimum conditioning humidity which ensures the safe transition of each hydrate-forming agent relevant to the mechanical stability of the tablet,
and optionally one or more other adjuvants.

The present invention further relates to a formulation (particularly a solid pharmaceutical formulation or composition, such as for example in the form of a tablet or tabletting material such as moist granulated material), comprising, obtained from or essentially consisting of:

one or more selectively dried hydrate-forming active substances and/or adjuvants (e.g. L-arginine, particularly as adjuvant), particularly those selectively dried hydrate-forming active substances and/or adjuvants obtained by drying to a point not below the relative humidity which corresponds to the phase transition of the hydrate-forming agent relevant to the mechanical stability of the tablet in the desorption cycle,
optionally one or more other active substances,
and optionally one or more other pharmaceutically acceptable adjuvants; in particular if the release of the water (particularly water of crystallisation) in the hydrate-forming agent and the change in the crystal lattice structure possibly associated therewith leads to a brittleness or mechanical instability of the tablet.

Moreover the present invention relates to a formulation (particularly a solid pharmaceutical formulation or composition, such as e.g. in the form of a tablet or tabletting material, such as e.g. made up of wet granulated material) comprising, obtained from or essentially consisting of:

one or more selectively dried hydrate-forming active substances, particularly obtained by drying to a point not below a maximum drying humidity, which corresponds to the phase transition of the hydrate-forming agent relevant to the mechanical stability of the tablet,
optionally one or more other active substances,
and optionally one or more adjuvants.

Moreover the present invention relates to a formulation (particularly a solid pharmaceutical formulation or composition, such as e.g. in the form of a tablet or tabletting material, such as e.g. made up of wet granulated material) comprising, obtained from or essentially consisting of:

one or more active substances,
one or more selectively dried hydrate-forming adjuvants, particularly obtained by drying to a point not below a maximum drying humidity, which corresponds to the phase transition of the hydrate-forming agent relevant to the mechanical stability of the tablet,
and optionally one or more other adjuvants.

Moreover the present invention provides for the use of at least one hydrate-forming active substance and/or adjuvant (e.g. L-arginine, particularly as adjuvant) in a formulation (particularly a solid pharmaceutical formulation or composition, such as e.g. in the form of a tablet or tabletting material, such as e.g. comprising dry granulated material, dry compacted material or powder mixture), which has been moisture-conditioned at least to the relative humidity of its phase transition in the sorption cycle.

Moreover the present invention provides for the use of at least one hydrate-forming active substance and/or adjuvant (e.g. L-arginine, particularly as adjuvant) in a formulation (particularly a solid pharmaceutical formulation or composition, such as e.g. in the form of a tablet or tabletting material, e.g. made up of wet granulated material) which has not been dried below the relative humidity of its phase transition in the desorption cycle.

Moreover the present invention provides a method for preventing the incorporation of water (particularly water of crystallisation) and the change in the crystal lattice spacings possibly associated therewith in at least one hydrate-forming active substance and/or adjuvant (e.g. L-arginine, particularly as adjuvant) in a solid pharmaceutical formulation, (such as e.g. in all kinds of tablets), the method comprising the use of the hydrate-forming active substance and/or adjuvant (e.g. L-arginine) within the formulation in a selectively moisture-adjusted form according to the invention or in its hydrate form that is relevant to the mechanical stability of the formulation, which can be produced by setting an equilibrium humidity at the phase transition of the hydrate forming agent using a method as described herein (e.g. by moisture conditioning or drying).

Moreover the present invention provides a method for improving the hardness, the physical or mechanical stability, the durability and/or shelf life of a formulation containing at least one hydrate-forming active substance and/or adjuvant (e.g. L-arginine, particularly as adjuvant) (particularly a solid pharmaceutical formulation or composition, such as e.g. in the form of a tablet), the method comprising the use of the hydrate-forming active substance and/or adjuvant (e.g. L-arginine) within the formulation in a selectively moisture-adjusted or dried form according to the invention or in its hydrate form that is relevant to the mechanical stability of the formulation, which can be produced by setting an equilibrium humidity at the phase transition of the hydrate forming agent using a method as described herein (e.g. by moisture conditioning or drying).

In addition, the present invention relates to the use of a hydrate-forming active substance and/or adjuvant (e.g. L-arginine, particularly as adjuvant) within a formulation (particularly a solid pharmaceutical formulation or composition, such as e.g. in the form of a tablet or tabletting material, e.g. comprising dry granulated material, dry compacted material or powder mixture), wherein the hydrate-forming active substance and/or adjuvant is present in a selectively moisture-adjusted or dried form according to the invention or in its hydrate form that is relevant to the mechanical stability of the formulation, which can be produced by setting an equilibrium humidity at the phase transition of the hydrate forming agent using a method as described herein (e.g. by moisture conditioning or drying), in order to improve the hardness, physical stability, durability and/or shelf life of the formulation.

Moreover the present invention relates to the use of a hydrate-forming active substance and/or adjuvant (e.g. L-arginine, particularly as adjuvant) in a selectively moisture-adjusted or dried form according to the invention or in its hydrate form that is relevant to the mechanical stability of the formulation, which can be produced by setting an equilibrium humidity at the phase transition of the hydrate forming agent using a method as described herein (e.g. by moisture conditioning or drying), and optionally one or more other active substances and/or other adjuvants, for preparing a formulation (particularly a solid pharmaceutical formulation or composition, such as e.g. in the form of a tablet), particularly with improved hardness, physical stability, durability and/or shelf life.

Moreover the present invention relates to a method for preparing a formulation (particularly a solid pharmaceutical formulation or composition, such as e.g. in the form of a tablet or tabletting material), comprising the use of a hydrate-forming active substance and/or adjuvant (e.g. L-arginine, particularly as adjuvant) in a selectively moisture-adjusted or dried form according to the invention, or in its hydrate form relevant to the mechanical stability of the formulation, which can be produced by setting an equilibrium humidity at the phase transition of the hydrate forming agent using a method as described herein (e.g. by moisture conditioning or drying).

Moreover the present invention relates to a method for preparing a formulation (particularly a solid pharmaceutical formulation or composition, such as e.g. in the form of a tablet), comprising the use of a tabletting material or a final mixture containing one or more hydrate-forming active substances and/or adjuvants (e.g. L-arginine, particularly as adjuvant) in a selectively moisture-adjusted or dried form according to the invention or in their hydrate forms that are relevant to the mechanical stability of the formulation, which can be produced by providing an equilibrium humidity (e.g. by moisture-conditioning or drying according to the invention), that ensures each phase transition of the hydrate forming agents relevant to the mechanical stability of the formulation.

Moreover the present invention relates to a method for preparing a formulation (particularly a solid pharmaceutical formulation or composition, such as e.g. in the form of a tablet or tabletting material), comprising mixing a hydrate-forming active substance and/or adjuvant (e.g. L-arginine, particularly as adjuvant) with optionally one or more other active substances and/or optionally one or more other adjuvants, wherein the hydrate-forming active substance and/or adjuvant is present in a selectively moisture-adjusted or dried form according to the invention or in its hydrate form relevant to the mechanical stability of the formulations, which can be produced by providing an equilibrium humidity (e.g. by moisture-conditioning or drying according to the invention), that ensures the phase transition of the hydrate forming agent relevant to the mechanical stability of the formulation.

Moreover the present invention relates to a method for preparing a formulation (particularly a solid pharmaceutical formulation or composition, such as e.g. in the form of a tablet or tabletting material), comprising mixing a hydrate-forming active substance and/or adjuvant (e.g. L-arginine, particularly as adjuvant) with optionally one or more other active substances and/or optionally one or more other adjuvants, and providing an equilibrium humidity (e.g. by moisture-conditioning or drying according to the invention) before or after tabletting, that ensures the phase transition of the hydrate forming agent relevant to the mechanical stability of the formulation.

Moreover the present invention relates to a method for preparing a formulation (particularly a solid pharmaceutical formulation or composition, such as e.g. in the form of a tablet or tabletting material), comprising the step of controlled moisture adjustment (e.g. by moisture conditioning or by drying) of one or more hydrate-forming active substances and/or adjuvants used or present (e.g. L-arginine, particularly as adjuvant), e.g. by providing an equilibrium humidity (e.g. by moisture-conditioning or drying according to the invention) before or after tabletting, that ensures each phase transition of the hydrate forming agents relevant to the mechanical stability of the formulation.

Moreover the present invention relates to a method for preparing a formulation (particularly a solid pharmaceutical formulation or composition, such as e.g. in the form of a tablet, containing at least one hydrate-forming active substance and/or adjuvant, particularly containing L-arginine, relevant to the mechanical stability of the tablets) particularly with improved hardness, physical stability, durability and/or shelf life, comprising the step of controlled moisture adjustment (e.g. moisture conditioning or drying) of the formulation, the tabletting material or the final mixture containing the hydrate-forming active substance and/or adjuvant (alternatively the individual components or mixtures of the individual components thereof; particularly containing L-arginine), optionally before or after tabletting.

Moreover the present invention relates to a method for preparing a formulation (particularly a solid pharmaceutical formulation, or composition, such as e.g. in the form of a tablet or tabletting material), e.g. comprising dry granulated material, dry compacted material or powder mixture, containing one or more hydrate-forming active substances and/or adjuvants (e.g. L-arginine, particularly as adjuvant) and optionally one or more other active substances and/or optionally one or more other adjuvants, characterised in that one or more hydrate-forming active substances and/or adjuvants (or dry granulated material, dry compacted material or powder mixture containing it) are selectively moisture-conditioned,

particularly characterised in that the hydrate-forming active substances and/or adjuvants are selectively moisture-conditioned such that the moisture adjustment (moisture conditioning, e.g. of the individual components or of a mixture of the individual components, of the tabletting material present (e.g. dry granulated material, dry compacted material or powder mixture), of the final mixture or of the formulation) ensures that the hydrate-forming active substances and/or adjuvants are present in their hydrate forms that are relevant to the mechanical stability of the formulation (in the case of the hydrate forming agent L-arginine for example a minimum conditioning humidity of ≧38-40% r.h./25° C. is needed so that L-arginine exceeds its phase transition in the sorption cycle);
particularly if the uptake or incorporation of the water (particularly water of crystallisation) in the hydrate forming agent and the change in the crystal lattice structure possibly associated therewith leads to brittleness or mechanical instability of the formulation (e.g. tablet).

Moreover the present invention relates to a method for preparing a formulation (particularly a solid pharmaceutical formulation, or composition, such as e.g. in the form of a tablet or tabletting material), e.g. of wet granulated material, containing one or more hydrate-forming active substances and/or adjuvants (e.g. L-arginine, particularly as adjuvant) and optionally one or more other active substances and/or optionally one or more other adjuvants, characterised in that one or more hydrate-forming active substances and/or adjuvants (or the wet granulated material containing them) are selectively dried,

particularly characterised in that the hydrate-forming active substances and/or adjuvants are selectively dried such that the moisture adjustment (moisture conditioning, e.g. of the individual components or of a mixture of the individual components, of the tabletting material present (e.g. wet granulated material), of the final mixture or of the formulation) ensures that the hydrate-forming active substances and/or adjuvants are present in their hydrate forms that are relevant to the mechanical stability of the formulation (in the case of the hydrate forming agent L-arginine for example the humidity must not fall below a maximum drying humidity of 30% r.h./25° C. so that L-arginine does not fall short of its phase transition in the desorption cycle);
particularly if the uptake or incorporation of the water (particularly water of crystallisation) in the hydrate forming agent and the change in the crystal lattice structure possibly associated therewith leads to mechanical instability of the formulation (e.g. tablet).

Moreover the present invention relates to a method for preparing a formulation (particularly a solid pharmaceutical formulation, or composition, such as e.g. in the form of a tablet or tabletting material), e.g. using dry granulated material, dry compacted material or powder mixture, containing one or more hydrate-forming active substances and/or adjuvants (e.g. L-arginine, particularly as adjuvant) and optionally one or more other active substances and/or optionally one or more other adjuvants, wherein the one or more hydrate-forming active substances and/or adjuvants are present in selectively moisture-conditioned form, which is obtained by moisture conditioning (e.g. of the formulation, of the tabletting material, of the dry granulated material, of the dry compacted material, of the powder mixture or the final mixture, particularly each containing the hydrate-forming active substance and/or adjuvant, or the individual components or mixtures of the individual components; particularly containing the relevant hydrate-forming active substance and/or adjuvant; particularly containing L-arginine) with at least the minimum conditioning that ensures the phase transition of each of each hydrate forming agent relevant to the mechanical stability of the tablet in the sorption cycle (e.g. ≧38-40% r.h./25° C. minimum conditioning humidity for L-arginine).

Moreover the present invention relates to a method for preparing a formulation (particularly a solid pharmaceutical formulation, or composition, such as e.g. in the form of a tablet or tabletting material), e.g. using wet granulated material, containing one or more hydrate-forming active substances and/or adjuvants (e.g. L-arginine, particularly as adjuvant) and optionally one or more other active substances and/or optionally one or more other adjuvants, wherein the one or more hydrate-forming active substances and/or adjuvants are in selectively dried form, which is obtained by drying (e.g. the formulation, the tabletting material, the wet granulated material or the final mixture, particularly each containing the hydrate-forming active substance and/or adjuvant, or the individual components or the mixtures of the individual components; particularly containing the relevant hydrate-forming active substance and/or adjuvant; particularly containing L-arginine), to a point not below the maximum drying humidity, which corresponds to the phase transition of the hydrate forming agent relevant to the mechanical stability of the tablet in the desorption cycle (e.g. not <30% r.h./25° C. maximum drying humidity for L-arginine).

Accordingly, the present invention further relates to a method for preparing a formulation (particularly a solid pharmaceutical formulation or composition, such as e.g. in the form of a tablet, containing at least one hydrate-forming active substance and/or adjuvant relevant to the mechanical stability of the tablets, particularly containing L-arginine) with improved hardness, physical stability, durability and/or shelf life, comprising combining the components and the step according to the invention of a systematic moisture conditioning of the formulation, of the tabletting material (e.g. dry granulated material, dry compacted material or powder mixture) or of the final mixture, particularly each containing the hydrate-forming active substance and/or adjuvant (alternatively the individual components or mixtures of the individual components thereof; particularly containing the relevant hydrate-forming active substance and/or adjuvant, particularly containing L-arginine), particularly before the tabletting.

Moreover the present invention relates to a method for preparing a formulation (particularly a solid pharmaceutical formulation or composition, such as e.g. in the form of a tablet, containing at least one hydrate-forming active substance and/or adjuvant relevant to the mechanical stability of the tablets, particularly containing L-arginine) with improved hardness, physical stability, durability and/or shelf life, comprising combining the components and the step according to the invention of a systematic drying of the formulation, of the tabletting material (e.g. wet granulated material) or of the final mixture, particularly each containing the hydrate-forming active substance and/or adjuvant (alternatively the individual components or mixtures of the individual components thereof; particularly containing the relevant hydrate-forming active substance and/or adjuvant, particularly containing L-arginine), particularly before the tabletting.

Moreover the present invention relates to the formulation (particularly solid pharmaceutical formulations or compositions, e.g. in the form of tablets or tablet mixtures, particularly containing L-arginine) which may be or are obtained by one of the (manufacturing) methods described herein.

Hydrate-forming components or mixtures or compositions containing hydrate-forming components may be adjusted in controlled manner to a relative humidity at which the phase transition relevant to the subsequent mechanical stability of the tablets is exceeded, for example by conditioning them in open warehouses or by storing in a moisture-permeable packaging at a certain climate (defined temperature and relative humidity, corresponding to the relevant phase transition of the hydrate forming agent). Alternatively, these materials which are moisture-conditioned to their phase transition may also for example be produced by mixing strongly moisture-laden or saturated material with dry material. From the sorption capacity of the material depending on the relative humidity and temperature provided, the mixing ratio of saturated and dry material can be calculated which yields the desired relative equilibrium humidity of the mixture at a specific temperature, i.e. the conditioning humidity for successfully exceeding the relevant phase transition of the hydrate-forming agent in the material.

Example of the preparation of moisture-conditioned tabletting material:

Moisture-conditioned tabletting material, e.g. containing the hydrate forming agent L-arginine, may be prepared, for example, by

a) Storing the finished end mixture either open or in a moisture-permeable outer packaging under defined constant climatic conditions (e.g. 25° C., at least 38 to 40% r.h) while at the same time monitoring the relative humidity and temperature. The defined climatic conditions may be produced either in a climate controlled enclosure or climate controlled chamber or by means of saturated saline solutions in a sealed container (e.g. desiccator) which is stored in an environment having a defined temperature (e.g. climate controlled enclosure or chamber). Alternatively, the saturated saline solution may also be replaced by silica gel which has previously been selectively moisture-conditioned. There is also the possibility of introducing a moist air current into a container, the water content being adjusted by means of the partial nitrogen pressure using a water reservoir.
b) Alternatively, the conditioning may also be carried out solely on the critical component such as L-arginine, for example. For this purpose this component is stored as described under a) and after a sufficient conditioning time (e.g. by monitoring the time when constant mass is reached e.g. at 25° C. and at least 38-40% r.h. or higher in the sorption cycle) it is added to the other components. The degree of conditioning humidity above 38-40% r.h./25° C. is dependent on the sorption isotherms and the existing water content in the other components. The equilibrium humidity achieved in the tabletting material can be calculated and in the case of the hydrate-forming agent L-arginine contained therein it must not fall below an equilibrium humidity of 30% r.h./25° C., since at <30% r.h./25° C. the phase transition of L-arginine takes place in the desorption cycle (c.f. FIG. 1). The advantage of this conditioning variant is, among other things, that compared with the volume of the final mixture very much smaller amounts of powdered material need to be stored for the conditioning, thus saving on storage capacity. Moreover, moisture-sensitive components of a formulation may in this way be protected until just before the tabletting process by not being exposed to the moisture conditioning.
c) In an alternative process, reconditioning may be carried out in the total powder mixture for compacting: dry material, e.g. L-arginine, may absorb moisture from other components (e.g. adjuvants or active substances) which release water under the storage conditions used, depending on their sorption isotherms, so that, finally, the desired equilibrium humidity is achieved with the phase transition being exceeded in the case of L-arginine. This variant tends to represent an exception, as the individual components (active substances and/or adjuvants) are normally supplied with low moisture content on the basis of their chemical, physical and microbial long-term storage stability. The other components should therefore initially be moisture-conditioned in order to be stored for a further period of time after the addition of the L-arginine to adjust the humidity equilibrium.

The teaching according to the invention (e.g. moisture conditioning, drying, etc.) may for example be applied as described herein to a composition (particularly containing L-arginine) or formulation (particularly a solid pharmaceutical formulation, mixture, final mixture, tabletting material or preparation, e.g. in the form of a tablet), for example to the following embodiments:

In one embodiment of the present invention the active substances mentioned herein comprise DPP-4 inhibitors, preferably an orally active DPP-4 inhibitor with an amino group, particularly a free or primary amino group.

Examples of DPP-4 inhibitors include linagliptin, vildagliptin, saxagliptin or alogliptin.

A preferred active substance (DPP-4 inhibitor) for the purposes of the invention is linagliptin.

In another embodiment of the present invention, the active substances mentioned herein include biguanides (e.g. metformin such as metformin hydrochloride), thiazolidinedione (e.g. pioglitazone such a pioglitazone hydrochloride), statins (e.g. atorvastatin) or ARBs (e.g. telmisartan).

In an embodiment of the present invention, one of the active substances mentioned herein is metformin such as metformin hydrochloride.

In an embodiment of the present invention, one of the active substances mentioned herein is linagliptin.

In an embodiment of the present invention, one of the adjuvants mentioned herein is L-arginine.

In a further embodiment of the present invention, an active substance mentioned herein is linagliptin and/or metformin (such as metformin hydrochloride) and one of the adjuvants mentioned herein is L-arginine.

In one particular embodiment of the present invention one of the active substances mentioned herein is linagliptin and one of the adjuvants mentioned herein is L-arginine.

In another embodiment of the present invention the active substance (e.g. DPP-4 inhibitor, particularly linagliptin) and the adjuvant L-arginine are present in a ratio by weight of about 1:20 to about 10:1 or from about 1:15 to about 10:1, preferably from about 1:10 to about 10:1 or, for example, from about 1:2 to about 5:1, in the formulations or compositions according to the invention.

In another embodiment of the present invention the one or more active substances mentioned herein are linagliptin and optionally another active substance selected from among metformin (e.g. metformin hydrochloride), pioglitazone (e.g. pioglitazone hydrochloride), atorvastatin and telmisartan, and one of the adjuvants mentioned herein is L-arginine.

In a particular embodiment of the present invention the active substances mentioned herein comprise linagliptin and metformin (particularly linagliptin in combination with metformin hydrochloride), and the adjuvants mentioned herein comprise L-arginine.

In another particular embodiment of the present invention the active substances mentioned herein comprise linagliptin and pioglitazone (particularly linagliptin in combination with pioglitazone hydrochloride), and the adjuvants mentioned herein comprise L-arginine.

In another embodiment of the present invention the adjuvants mentioned herein may optionally comprise in addition to L-arginine other adjuvants such as e.g. one or more fillers, one or more diluents, one or more binders, one or more lubricants, one or more release agents, one or more disintegrants, one or more breakdown agents, one or more flow agents, one or more coating agents, one or more plasticisers, one or more pigments, etc.

In another embodiment of the present invention the one or more active substances mentioned herein are linagliptin and optionally another active substance selected from among metformin (e.g. metformin hydrochloride) and pioglitazone (e.g. pioglitazone hydrochloride), and the one or more adjuvants mentioned herein are L-arginine (particularly as stabiliser) and optionally one or more other adjuvants e.g. selected from among one or more fillers (e.g. D-mannitol, maize starch and/or pre-gelatinised starch), a binder (e.g. copovidone), optionally a lubricant (e.g. magnesium stearate), optionally a release agent and optionally a flow agent (e.g. anhydrous colloidal silicon dioxide).

In another embodiment of the present invention the one or more active substances mentioned herein are linagliptin and optionally another active substance selected from metformin (e.g. metformin hydrochloride, e.g. in immediate release formulation or in extended release formulation), and the one or more adjuvants mentioned herein are L-arginine (particularly as stabiliser) and optionally one or more other adjuvants e.g. selected from among one or more fillers (e.g. D-mannitol, maize starch and/or pre-gelatinised starch), a binder (e.g. copovidone), optionally a lubricant (e.g. magnesium stearate), optionally a release agent and optionally a flow agent (e.g. anhydrous colloidal silicon dioxide).

In another embodiment of the present invention the one or more active substances mentioned herein are linagliptin (e.g. in an amount of 0.5, 1, 2.5, 5 or 10 mg) and optionally another active substance selected from metformin (e.g. metformin hydrochloride, e.g. in an amount of 250, 500, 625, 750, 850 or 1000 mg), and the one or more adjuvants mentioned herein are L-arginine (particularly as stabiliser, e.g. in an amount of 0.5 to 50 mg, e.g. 1 to 50 mg, preferably 1 to 25 mg, or e.g. 0.5 to 10 mg) and optionally one or more other adjuvants selected from one or more fillers (e.g. D-mannitol, maize starch and/or pre-gelatinised starch), a binder (e.g. copovidone), a lubricant (e.g. magnesium stearate), and a flow agent (e.g. anhydrous colloidal silicon dioxide).

In another embodiment of the present invention the one or more active substances mentioned herein are linagliptin (e.g. in an amount of 2.5 mg, particularly in immediate release formulation) and optionally another active substance selected from metformin (particularly metformin hydrochloride, e.g. in an amount of 500, 850 or 1000 mg, particularly in immediate release formulation), and the one or more adjuvants mentioned herein contain L-arginine (particularly as stabiliser, e.g. in an amount of 1 to 50 mg, preferably 1 to 25 mg), and optionally a filler (e.g. maize starch), optionally a binder (e.g. copovidone), optionally a lubricant (e.g. magnesium stearate) and optionally a flow agent (e.g. anhydrous colloidal silicon dioxide).

Pharmaceutical compositions of this invention may comprise the following parts by weight—optionally in the part of the composition containing DPP-4 inhibitor—(% of the total weight of the part containing DPP-4 inhibitor):

| | |
|---|---|
| 0.2-10% | DPP-4 inhibitor (particularly linagliptin), and |
| 0.1-10% | L-arginine. |

Pharmaceutical compositions of this invention may contain the DPP-4 inhibitor (particularly linagliptin) and L-arginine in a ratio by weight from about 1:20 to about 10:1 or from about 1:15 to about 10:1 or from about 1:10 to about 10:1, particularly from 1:10 to 5:2, e.g. in a weight ratio of 1:10, 1:8.5, 1:5, 1:1, or 1:0.4, e.g. in a weight ratio of 2.5 mg:25 mg, 2.5 mg:21.2 mg, 2.5 mg:12.5 mg, 2.5 mg:2.5 mg, or 2.5 mg:1 mg.

Pharmaceutical compositions of this invention may contain metformin hydrochloride and L-arginine in a ratio by weight from about 40:1 to about 1000:1, e.g. in a weight ratio of 40:1, 200:1, 340:1, 400:1, 500:1, 850:1, or 1000:1, e.g. a weight ratio of 500 mg:12.5 mg, 850 mg:21.2 mg, 1000 mg:25 mg, 500 mg:2.5 mg, 850 mg:2.5 mg, 1000 mg:2.5 mg, 500 mg:1 mg, 850 mg:1 mg, or 1000 mg:1 mg.

Pharmaceutical compositions of this invention may contain the DPP-4 inhibitor (particularly linagliptin), metformin hydrochloride and L-arginine in a weight ratio of about 1:200:0.4 to about 1:200:5 (e.g. 1:200:0.4, 1:200:1, 1:200:5), or from about 1:340:0.4 to about 1:340:8.5 (e.g. 1:340:0.4, 1:340:1, 1:340:8.5), or from about 1:400:0.4 to about 1:400:10 (e.g. 1:400:0.4, 1:400:1, 1:400:10).

Pharmaceutical compositions of this invention may contain one or more of the following amounts (% of the total weight of the film-coated tablet):

| | |
|---|---|
| 0.1-0.5% | DPP-4 inhibitor (particularly linagliptin), |
| 47-85% | metformin HCl, |
| 0.07-2.2% | L-arginine, |
| 3.9-8.1% | binder (e.g. copovidone), |
| 2.3-5.9% | fillers 1 (e.g. maize starch), |
| 0-4.4% | fillers 2 (e.g. pre-gelatinised starch), |
| 0-33% | fillers 3 (e.g. D-mannitol), |
| 0.7-1.5% | lubricant (e.g. magnesium stearate), and |
| 0.1-0.5% | flow agent (e.g. anhydrous colloidal silicon dioxide). |

A wet granulated material according to the invention may for example contain or essentially consist of: a DPP-4 inhibitor (particularly linagliptin), metformin HCl, L-arginine (as adjuvant or stabiliser), a binder (e.g. copovidone), and optionally one or more fillers (e.g. maize starch, pre-gelatinised starch and/or mannitol).

Novel formulations, tabletting material or tablets according to this invention may for example contain or essentially consist of: a DPP-4 inhibitor (particularly linagliptin), metformin HCl, L-arginine (as adjuvant or stabiliser), a binder (e.g. copovidone), one or more fillers (e.g. maize starch, pre-gelatinised starch and/or mannitol), optionally a lubricant (e.g. magnesium stearate) and optionally a flow agent (e.g. anhydrous colloidal silicon dioxide).

In another embodiment of the present invention the tablets mentioned herein include for example single-layer, double-layer or triple-layer tablets, coated core tablets, film-coated tablets, etc.

In another embodiment of the present invention the teaching according to the invention (e.g. moisture conditioning, drying, etc.) may be applied to an L-arginine-containing composition or formulation (particularly a solid pharmaceutical formulation, mixture, final mixture, tabletting material, or preparation, such as e.g. in the form of a tablet) as follows, or the one or more active substances mentioned herein are linagliptin (e.g. in an amount of 2.5 mg, particularly in immediate release formulation) and another active substance selected from metformin (particularly metformin hydrochloride, e.g. in an amount of 500, 850 or 1000 mg, particularly in immediate release formulation), and the one or more adjuvants mentioned herein are L-arginine (particularly as stabiliser, e.g. in an amount of 1 to 50 mg, preferably 1 to 25 mg, e.g. in a suitable amount of about 12.5 mg, 21.2 mg or 25.0 mg) and a filler (e.g. maize starch, e.g. in a suitable amount of about 20.0 mg, 33.1 mg or 42.5 mg), a binder (e.g. copovidone, e.g. in a suitable amount of about 47.5 mg, 80.5 mg or 95.0 mg), a lubricant (e.g. magnesium stearate, e.g. in a suitable amount of about 5.0 mg, 8.5 mg or 10.0 mg), and a flow agent (e.g. anhydrous colloidal silicon dioxide, e.g. in a suitable amount of about 2.5 mg, 4.2 mg or 5.0 mg).

In another embodiment of the present invention the teaching according to the invention (e.g. moisture conditioning, drying, etc.) may be applied to a (particularly L-arginine-containing) composition or formulation (particularly a solid pharmaceutical formulation, mixture, final mixture, tabletting material, wet granulated material, layer, film, coating or preparation, e.g. in the form of a tablet) as described in WO 2009/121945 (the disclosure of which is incorporated herein by reference). In particular, mention should be made of the L-arginine-containing formulations and their preparation as described in Examples 1 to 4 of WO 2009/121945.

In another embodiment of the present invention the teaching according to the invention (e.g. moisture conditioning, drying, etc.) may be applied to a (particularly L-arginine-containing) composition or formulation (particularly a solid pharmaceutical formulation, mixture, final mixture, tabletting material, layer, film, coating or preparation, e.g. in the form of a tablet) as described in WO 2012/120040 or WO 2013/131967 (the disclosure of each of which is incorporated herein by reference).

In another embodiment of the present invention the formulations or compositions containing the DPP-4 inhibitors (particularly linagliptin) may be prepared by the skilled man using permitted formulation adjuvants by methods described in the prior art. Examples of such adjuvants are diluents, binders, carriers, fillers, lubricants, flow agents, crystallisation retardants, disintegrants, solubilisers, colourings, pH regulators, surfactants and/or emulsifiers.

Examples of suitable diluents include cellulose powder, calcium hydrogen phosphate, erythritol, (low-substituted) hydroxypropylcellulose, mannitol, pregelatinised starch or xylitol.

Examples of suitable binders include copolymers of vinylpyrrolidone with other vinyl derivatives (copovidone), hydroxypropylmethylcellulose (HPMC), hydroxypropylcellulose (HPC) polyvinylpyrrolidone (povidone), pregelatinised starch, or low-substituted hydroxypropylcellulose.

Examples of suitable lubricants include talc, polyethyleneglycol, calcium behenate, calcium stearate, hydrogenated castor oil or magnesium stearate.

Examples of suitable disintegrants include maize starch or crospovidone.

Suitable methods of preparing pharmaceutical formulations of the active substances (particularly containing a DPP IV inhibitor, particularly linagliptin) are
Direct tabletting in powder mixtures with suitable tabletting adjuvants;
Granulation with suitable adjuvants and subsequent mixing with suitable adjuvants and optionally subsequent tabletting as well as film coating; or
packing of powder mixtures or granules into capsules.

Suitable granulation methods for this purpose are
wet granulation in the intensive mixer followed by fluidised bed drying;
one-pot granulation;
fluidised bed granulation; or
dry granulation (e.g. by roller compaction) with suitable adjuvants and subsequent tabletting or packing into capsules.

Accordingly, in a further embodiment, the present invention relates to one or more of the following substances and/or adjuvants, or compositions or formulations thereof (e.g. tableting materials or tablets containing one or more of such substances and/or adjuvants), in moisture adjusted (e.g. moisture conditioned or dried) form: linagliptin, metformin (e.g. metformin hydrochloride) or pioglitazone (e.g. pioglitazone hydrochloride), and L-arginine (particularly as adjuvant or stabiliser), and optionally one or more other adjuvants, e.g. one or more fillers (e.g. D-mannitol, maize starch and/or pre-gelatinised starch), a binder (e.g. copovidone), optionally a lubricant (e.g. magnesium stearate), optionally a release agent and/or optionally a flow agent (e.g. anhydrous colloidal silicon dioxide). Moisture-adjustment (e.g. moisture conditioning or drying) of the substances and/or adjuvants, or compositions, formulations or mixtures thereof, may be obtained as described herein by way of example, or analogously or similarly thereto.

Further embodiments of the invention:

1. Formulation (particularly a solid pharmaceutical formulation, mixture, final mixture, tabletting material or preparation, or composition, e.g. in the form of a tablet) comprising one or more hydrate-forming active substances and/or adjuvants (e.g. L-arginine as adjuvant or as active substance, particularly as adjuvant), optionally together with one or more other active substances and/or adjuvants, characterised in that the said one or more hydrate-forming active substances and/or adjuvants are selectively moisture-conditioned or dried,
particularly characterised in that the said one or more hydrate-forming active substances and/or adjuvants are selectively moisture-conditioned or dried hydrate-forming active substances and/or adjuvants of the kind that are obtained by moisture adjustment (moisture conditioning or drying, e.g. of the individual components or of a mixture of the individual components, of the tabletting material, of the final mixture or of the formulation), which ensures that the hydrate-forming active substances and/or adjuvants are present in their in their hydrate forms that are relevant to the mechanical stability of the formulation;
particularly if the uptake or incorporation of the water (e.g. water of crystallisation, adsorption, swelling or capillary water, particularly water of crystallisation) in the hydrate forming agent and the change in the crystal lattice structure possibly associated therewith leads to brittleness or mechanical instability of the formulation (e.g. tablet), and/or
particularly if the release of the water (e.g. water of crystallisation, adsorption, swelling or capillary water, particularly water of crystallisation) in the hydrate forming agent and the change in the crystal lattice structure possibly associated therewith leads to mechanical instability of the formulation (e.g. tablet).
2. Formulation (particularly a solid pharmaceutical formulation or composition, e.g. in the form of a tablet) or tabletting material comprising or obtained from:
L-arginine as adjuvant or as active substance, particularly as adjuvant,
optionally one or more active substances,
and optionally one or more other adjuvants,
the L-arginine being in its hydrate form (e.g. L-arginine hydrate containing at least about 1.5 mol water of crystallisation/1 mol L-arginine).
3. Formulation according to embodiment 2, wherein the L-arginine is L-arginine that has been selectively moisture-conditioned to at least 38-40% r.h./25° C., particularly which has exceeded its phase transition in the sorption cycle.
4. Formulation according to embodiment 2, wherein the L-arginine is L-arginine that has been selectively dried to max. 30% r.h./25° C., particularly which has not fallen below its phase transition in the desorption cycle.
5. Use of a selectively moisture-conditioned hydrate-forming active substance and/or adjuvant, particularly use of selectively moisture-conditioned L-arginine particularly as adjuvant, preferably L-arginine obtainable by moisture conditioning with a minimum conditioning humidity of ≧38-40% r.h./25° C., and optionally one or more other adjuvants and/or active substances, for the preparation of or within a formulation (particularly a solid pharmaceutical formulation or composition, such as e.g. in the form of a tablet) particularly with improved hardness, physical or mechanical stability, durability and/or shelf-life, wherein the hydrate-forming agent or the L-arginine is in its hydrate form (e.g. L-arginine hydrate containing at least about 1.5 mol water of crystallisation/1 mol L-arginine).
6. Use of a selectively dried hydrate-forming active substance and/or adjuvant, particularly use of selectively dried L-arginine particularly as adjuvant, preferably L-arginine obtainable by drying to a point not below a maximum drying humidity of 30% r.h./25° C., and optionally one or more other adjuvants and/or active substances, for the preparation of or within a formulation (particularly a solid pharmaceutical formulation or composition, such as e.g. in the form of a tablet) particularly with improved hardness, physical or mechanical stability, durability and/or shelf-life, wherein the hydrate-forming agent or the L-arginine is in its hydrate form (e.g. L-arginine hydrate containing at least about 1.5 mol water of crystallisation/1 mol L-arginine).
7. Method for preventing the incorporation of water (particularly water of crystallisation) and the restructuring of the crystal lattice which may optionally take place as a result in a hydrate-forming active substance and/or adjuvant, particularly in L-arginine (particularly as adjuvant) in a formulation (particularly a solid pharmaceutical formulation or composition, such as e.g. in the form of a tablet), particularly the associated mechanical instability of the formulation, the method comprising the use of selectively moisture-conditioned active substance and/or adjuvant, particularly selectively moisture-conditioned L-arginine within the formulation, preferably selectively moisture-conditioned L-arginine of the kind that is obtainable by moisture conditioning with a minimum conditioning humidity of ≧38-40% r.h./25° C., the hydrate-forming agent or the L-arginine being in its hydrate form (e.g. L-arginine hydrate containing at least about 1.5 mol water of crystallisation/1 mol L-arginine).
8. Method for preventing the release of water (particularly water of crystallisation) and the restructuring of the crystal lattice which may optionally take place as a result in a hydrate-forming active substance and/or adjuvant, particularly in L-arginine (particularly as adjuvant) in a formulation (particularly a solid pharmaceutical formulation or composition, such as e.g. in the form of a tablet), particularly the associated mechanical instability of the formulation, the method comprising the use of selectively dried active substance and/or adjuvant, particularly selectively dried L-arginine within the formulation, preferably selectively dried L-arginine of the kind that is obtainable by drying to a point not below a maximum drying humidity of 30% r.h./25° C., the hydrate-forming agent or the L-arginine being in its hydrate form (e.g. L-arginine hydrate containing at least about 1.5 mol water of crystallisation/1 mol L-arginine).
9. Method for improving the hardness, the physical or mechanical stability, the durability and/or shelf-life of a formulation containing L-arginine (particularly a solid pharmaceutical formulation or composition, e.g. in the form of a tablet), the method comprising the use of selectively moisture-conditioned or selectively dried L-arginine within the formulation, the L-arginine being in its hydrate form (e.g. L-arginine hydrate containing at least about 1.5 mol water of crystallisation/1 mol L-arginine).
10. Method for preparing a formulation (particularly a solid pharmaceutical formulation or composition, such as e.g. in the form of a tablet), comprising mixing L-arginine (particularly as adjuvant) with one or more active substances and/or one or more adjuvants, the L-arginine being a selectively moisture-conditioned or selectively dried L-arginine, the L-arginine being in its hydrate form (e.g. L-arginine hydrate containing at least about 1.5 mol water of crystallisation/1 mol L-arginine).
11. The method according to embodiment 9 or 10, wherein the selectively moisture-conditioned L-arginine is obtainable by moisture conditioning with a minimum conditioning humidity of ≧38-40% r.h./25° C.
12. The method according to embodiment 9 or 10, wherein the selectively dried L-arginine is obtainable by drying to a point not below a maximum drying humidity of 30% r.h./25° C.
13. Method for preparing a formulation (particularly a solid pharmaceutical formulation or composition, such as e.g. in the form of a tablet, containing at least one hydrate-forming active substance and/or adjuvant relevant to the mechanical stability of the tablets, particularly containing L-arginine, particularly as adjuvant), e.g. a formulation according to one of embodiments 1 to 4, particularly a formulation with improved hardness, physical or mechanical stability, durability and/or shelf-life, (e.g. comprising dry granulated material, dry compacted material or powder mixture, or wet granulated material), comprising the step of systematic moisture conditioning or systematic drying of the formulation, of the tabletting material, of the final mixture, of the individual components or mixture of the individual components, such as containing the hydrate-forming agent or L-arginine, optionally before or after tabletting.
14. The method according to embodiment 13, wherein the hydrate-forming agent is L-arginine and the systematic moisture conditioning is carried out with a minimum conditioning humidity of ≧38-40% r.h./25° C.
15. The method according to embodiment 13, wherein the hydrate-forming agent is L-arginine and the systematic drying is carried out to a point not below a maximum drying humidity of 30% r.h./25° C.
16. The method according to embodiment 13, 14, or 15, wherein the systematic moisture conditioning or systematic drying is carried out so that the hydrate-forming agent obtained and/or the L-arginine obtained is in its hydrate form (e.g. L-arginine hydrate containing at least about 1.5 mol water of crystallisation/1 mol L-arginine).
17. The method according to embodiment 13, 14, 15 or 16, wherein the tabletting material is a dry granulated material, a dry compacted material or a powder mixture (e.g. as tabletting material for moisture conditioning).
18. The method according to embodiment 13, 14, 15 or 16, wherein the tabletting material is a wet granulated material (e.g. as tabletting material for drying).
19. Formulation, use or method according to at least one of the preceding embodiments, wherein the moisture-conditioned L-arginine can be prepared:
by open storage or storage of the L-arginine in a moisture-permeable package in a specific climate, or
by mixing highly moisture-laden or moisture-saturated L-arginine with one or more dry adjuvants and/or active substances,
or by mixing one or more moisture-laden adjuvants and/or active substances with dry L-arginine,
in order thus to obtain the systematic moisture conditioning of the L-arginine.
20. Formulation, use or method according to at least one of the preceding embodiments, wherein the selectively moisture-conditioned L-arginine is obtainable by moisture conditioning of L-arginine at at least 38% r.h., preferably above 40% r.h., at 25° C. (e.g. 45% r.h./25° C.).
21. Formulation (particularly a solid pharmaceutical formulation or composition, e.g. in the form of a tablet) obtainable or obtained by a method comprising the systematic moisture conditioning or drying of the formulation, of the tabletting material, of the final mixture, of one or more individual components or mixtures of components of the formulation comprising or essentially consisting of:
one or more active substances,
L-arginine particularly as adjuvant,
and optionally one or more other adjuvants.
22. Formulation according to embodiment 21, wherein the tabletting material or mixture is a dry granulated material, a dry compacted material or a powder mixture.
23. Formulation according to embodiment 21, wherein the tabletting material or mixture is a wet granulated material.
24. Formulation, use or method according to at least one of the preceding embodiments, wherein L-arginine is present as adjuvant or is used as adjuvant.
25. Formulation, use or method according to at least one of embodiments 1-24, wherein for the moisture conditioning comprising one or more hydrate-forming agents at least the minimum conditioning humidity is used at which all the hydrate-forming agents crucial to the mechanical stability of the tablet have exceeded their phase transition in the sorption cycle, which is causally responsible for the mechanical stability of the tablet.
26. Formulation, use or method according to at least one of embodiments 1-24, wherein for the drying comprising one or more hydrate-forming agents at most the maximum drying humidity is used at which the hydrate-forming agent crucial to the mechanical stability of the tablet has not yet fallen below its phase transition in the desorption cycle, which is causally responsible for the mechanical stability of the tablet.
27. Formulation, use or method according to at least one of the preceding embodiments, wherein the said one or more active substances are linagliptin and/or metformin (such as metformin HCl), particularly linagliptin in combination with metformin hydrochloride.
28. Formulation, use or method according to at least one of the preceding embodiments, wherein the said one or more active substances are linagliptin and/or pioglitazone (such as pioglitazone HCl), particularly linagliptin in combination with pioglitazone hydrochloride.
29. Formulation, use or method according to at least one of the preceding embodiments, wherein the said one or more active substances are linagliptin and optionally another active substance selected from metformin (e.g. metformin hydrochloride) and pioglitazone (e.g. pioglitazone hydrochloride), and the said one or more adjuvants are L-arginine (particularly as stabiliser) and optionally one or more other adjuvants selected from one or more fillers (e.g. D-mannitol, maize starch and/or pre-gelatinised starch), a binder (e.g. copovidone), a lubricant (e.g. magnesium stearate), a release agent and a flow agent (e.g. anhydrous colloidal silicon dioxide).
30. Formulation, use or method according to at least one of the preceding embodiments, wherein the said formulation, composition, preparation, tabletting material, mixture or final mixture contains or essentially consists of: a DPP-4 inhibitor (particularly linagliptin), metformin HCl, L-arginine (as adjuvant or stabiliser), a binder (e.g. copovidone), one or more fillers (e.g. maize starch, pre-gelatinised starch and/or mannitol), optionally a lubricant (e.g. magnesium stearate), optionally a release agent and optionally a flow agent (e.g. anhydrous colloidal silicon dioxide).

### EXAMPLES

The following Examples are intended to illustrate the invention without restricting it in any way.

Tests were carried out on an L-arginine-containing test formulation of linagliptin and pioglitazone hydrochloride (pioglitazone HCl) on the basis of a selectively moisture-conditioned dry compacted material, to illustrate the invention.

The test formulation used by way of example consists of linagliptin, pioglitazone HCl, mannitol ParTech M200, finely powdered mannitol, L-arginine, crospovidone and magnesium stearate. From these formulation ingredients a dry compacted material was prepared and processed to form a final mixture. This final mixture was conditioned as a flat powder bed for 3 months in a climate-controlled enclosure at 45% r.h./25° C. until a constant mass was obtained and during this time it was circulated once a week to ensure uniform moistening. Towards the end of the conditioning period, the moisture content of the dry compacted material was checked again several times by pouching a partial quantity in an aluminium pouch together with a calibrated data logger. Before the tabletting an amount of 0.5 g of dry magnesium stearate per 99.5 g of dry compacted material was sieved in through a hand-held sieve with a mesh size of 144, and the mixture was combined in a Turbula mixer for 1 min. This was necessary to ensure frictionless tabletting. It is possible that the lubricating function of magnesium stearate is decreased on the absorption of moisture and thus in a preferred procedure the magnesium stearate is only added after the moisture conditioning of the mixture has taken place. The finished tablets were then immediately transferred into tightly sealable screw-top bottles to maintain the tablet moisture content of 45% r.h./25° C.

The results described hereinafter relate to the test formulation investigated on linagliptin+pioglitazone HCl by way of example (see above). The results may be generalised to other substances and formulations on the bases of the teaching contained herein.

The result of the stability investigations is that tablets produced from the L-arginine-containing tabletting material used here and moisture-conditioned according to the invention are mechanically stable over a storage period of 2.5 years. In particular, the breaking strength values that remain constant over this period lead one to conclude that in all probability the mechanical stability of the tablets is maintained even over the desired storage periods of 3 years.

After the preparation of tablets from tabletting material pre-conditioned at 25° C./45% r.h. samples thereof were stored at very different relative humidity levels for stability investigations. It was found that these tablets remain mechanically stable in spite of the wide range of different storage conditions. This is discussed below with reference to FIG. 1.

### Case 1:

The storage of tablets above the conditioning and tabletting humidity level (>38% r.h.-40% r.h., test formulation: 45% r.h. at 25° C.) shows that these tablets remain mechanically stable even at rel. humidity levels up to about 83% r.h. In the present Example this would mean that a product that originally becomes brittle within a day remains mechanically stable as a result of being prepared by the method proposed according to the invention, even when packaged in very moisture-permeable blister systems such as PVC, for example, and at the same time can be packaged very inexpensively.

### Case 2:

Test at relative humidity levels showed that tablets remain mechanically stable even below the tabletting humidity level (see above) (see FIG. 1, between >30 and 40% r.h. at 25° C.). In this part of the desorption cycle the hydrate form of L-arginine is stably maintained and only releases water of crystallisation from about <30% r.h./25° C. This is particularly important in connection with the objective of achieving the largest possible complete humidity range in which the stability of the tablet can be ensured.

### Case 3:

In addition, it was found that in this Example the relative humidity may also assume values of <30% r.h. at 25° C. and the product may even be subjected to very intensive drying in the glove box to humidity levels of around 0% r.h. Although a phase transition takes place at which L-arginine releases water of crystallisation and its crystal structure is changed, the mechanical stability of the tablets is retained. This should be particularly emphasised against the background that according to publications in the literature, in many hydrate-forming agents, once the hydration water has been released, the crystal structure may collapse and change into an amorphous state, which means that destruction of the tablets may be caused by this process.

### Case 4:

Within the scope of the experiments carried out investigations took place to discover how sharply fluctuating humidity levels affect the tablets produced by the method according to the invention. It was found that the tablets remain mechanically stable even under these conditions. It was further found that tablets remain mechanically stable even after passing through the phase transition several times.

To sum up, it is found that tablets can be produced from the dry compacted material formulation used here which has been moisture-preconditioned with at least 38-40% r.h. (at 25° C.), these tablets not only being mechanically stable over long periods but also remaining stable throughout the entire humidity range and thus having optimum characteristics for processing, packaging and in-use time. L-arginine-containing placebo mixtures and their stability as a function of moisture conditioning:

For further illustration of the effect according to the invention the following result of the storage of tablets produced from L-arginine-containing placebo mixture which has been subjected to different moisture conditioning processes may be used (see Photo 1):

### Storage: 83% r.h./23° C.

Storage period: 16 hours
Composition of an L-arginine-containing placebo mixture, e.g.:

### Mannitol ParTech M200: 74.5%

Magnesium stearate: 1.5%
Finely powdered mannitol: 9.5%

### L-arginine: 9.5%

### Crospovidone: 3.75%

Methylene blue (optional): 1.25%.

Generally, tabletting material may be obtained from an L-arginine-containing mixture by mixing all the components (with the exception of the lubricant such as magnesium stearate) in a Turbula mixer, for example, then adding the lubricant (e.g. magnesium stearate) and completing the mixing. Optionally a dry compacted material may also be produced as an alternative (e.g. as described above). Then the tabletting mixture is moisture-conditioned (e.g. in thin layers, e.g. under the conditions described herein, e.g. for about 3 months) and then tabletted. In a preferred procedure the addition of the lubricant (e.g. magnesium stearate) does not take place until after the moisture-conditioning of the mixture has been carried out (see above).

Mechanical stability of tablets of L-arginine-containing placebo mixtures with different moisture pre-conditioning (40% r.h. as against 33% r.h. moisture conditioning):

Conditioning at 40% r.h.: The tabletting material was initially conditioned at 40% r.h./25° C. until the humidity equilibrium was achieved and then tabletted. When stored above the phase transition of L-arginine (in this case 83% r.h./23° C.) these tablets did not show any fracturing. This mechanical stability remained for an observation period of 2.5 years in all.
Conditioning at 33% r.h.: The tabletting material was conditioned below the phase transition of L-arginine at 33% r.h./25° C. until the humidity equilibrium was achieved and then tabletted. Consequently the hydration of L-arginine in this case does not take place until after the tablets have been stored at 83% r.h./23° C. The phase transition that takes place in the tablet leads to total destruction of the product after only 16 hours.

### Other Fields of Application

The procedure according to the invention may also be used in other fields of application:

The method of systematic moisture conditioning of L-arginine according to the invention or the use of selectively moisture-conditioned material as a possible solution to the production of mechanically stable L-arginine-containing tablets from dry granulated material, dry compacted material or powder mixtures can be applied in principle to any formulation containing L-arginine. The rationale for this is the causal connection between mechanical stability and hydration at phase transition. Moreover, the method according to the invention is not particularly restricted to L-arginine but may theoretically be applied to any hydrate-forming adjuvant or active substance.

The procedure proposed here in the preconditioning and tabletting can be determined by the particular position of the phase transitions for hydration and dehydration. This should preferably be done by sorption experiments on the final mixture ready for tabletting, as the time taken to reach the humidity equilibrium at the critical phase transition may be strongly influenced (i.e. usually delayed) by other formulation components.

The method according to the invention may additionally be extended to wet granulated materials. If for example both mechanical and chemical, physical, pharmaceutical or microbial stability is determined by water, it may be necessary to dry a wet granulated material, (or a mixture, final mixture or tablet containing a wet granulated material) so that it is as dry as possible within the processing parameters. The method according to the invention may be used to define the lower humidity limit of the granulated material in the desorption cycle by means of the dehydration limits of adjuvants or active substances. For wet granulated materials containing L-arginine, for example, before tabletting the material must be dried at most to humidity levels of 30% r.h./25° C. since otherwise water (particularly water of crystallisation) will be released and tablets made from this material would become brittle.

Theoretically it is possible to apply the method to substances which bind water in other ways than as hydration water (also referred to as water of crystallisation), particularly in the form of adsorption, swelling or capillary water. By pre-conditioning such materials the damage to the product induced by wet adsorption after tabletting can be prevented. This option may be applied, for example, to the wet conditioning of swelling or disintegrating agents in tablet production for the systematic adjustment of the mechanical properties (e.g. hardness) of the tablets.

1. A solid pharmaceutical formulation or composition comprising one or more hydrate-forming active substances and/or adjuvants,
and, optionally, one or more other active substances and/or adjuvants,
characterised in that the said one or more hydrate-forming active substances and/or adjuvants are selectively moisture-conditioned or dried.

2. The solid pharmaceutical formulation or composition according to claim 1, wherein a said hydrate-forming active substance and/or adjuvant comprises L-arginine.

3. The solid pharmaceutical formulation or composition according to claim 1, wherein one or more of the hydrate-forming active substances and/or adjuvants are selectively moisture-conditioned or dried by:
moisture conditioning or drying of the individual components, or
of a mixture of the individual components, the tabletting material, the final mixture, or the formulation or composition.

4. The solid pharmaceutical formulation or composition according to claim 1, wherein the one or more of the hydrate-forming active substances and/or adjuvants are selectively moisture-conditioned or dried which ensures that the hydrate-forming active substances and/or adjuvants are present in their hydrate forms that are relevant to the mechanical stability of the formulation.

5. A solid pharmaceutical formulation or composition comprising or obtained from one or more active substances and L-arginine (particularly as first adjuvant), and, optionally one or more other adjuvants, wherein the L-arginine is in a hydrate form.

6. The formulation or composition according to claim 5, wherein the L-arginine in hydrate form contains at least about 1.5 mol water of crystallisation/1 mol L-arginine.

7. The formulation or composition according to claim 5, wherein the L-arginine has been selectively moisture-conditioned to at least 38-40% r.h./25° C.

8. The formulation or composition according to claim 5, wherein the L-arginine has been selectively dried to max. 30% r.h./25° C.

9. The formulation or composition according to claim 5, wherein the L-arginine has been selectively moisture-conditioned by:
open storage or storage of the L-arginine in a moisture-permeable package in a specific climate, or
mixing highly moisture-laden or moisture-saturated L-arginine with one or more dry adjuvants and/or active substances, or
mixing one or more moisture-laden adjuvants and/or active substances with dry L-arginine.

10. The formulation or composition according to claim 9, wherein the selectively moisture-conditioned L-arginine is obtained by moisture conditioning of L-arginine at at least 38% r.h at 25° C.

11. A solid pharmaceutical formulation, composition or tablet consisting essentially of one or more active substances and/or L-arginine, wherein the formulation, composition or tablet is prepared from a mixture or tabletting material and wherein, the process of preparing the formulation, composition or tablet comprises the systematic moisture conditioning or drying of:
(i) the formulation, composition or tablet,
(ii) the mixture or tabletting material,
(iii) the one or more active substances, and/or
(iv) L-arginine.

12. The formulation, composition or tablet according to claim 11, wherein the tabletting material or mixture is a dry granulated material, a dry compacted material or a powder mixture.

13. The formulation, composition or tablet according to claim 11, wherein the tabletting material or mixture is a wet granulated material.

14. The formulation, composition or tablet according to claim 11, which consists essentially of one or more active substances and L-arginine as first adjuvant.

15. The formulation, composition or tablet according to claim 11, wherein the said one or more active substances is selected from the group consisting of linagliptin and metformin.

16. The formulation, composition or tablet according to claim 11, wherein the said one or more active substances is selected from the group consisting of linagliptin and pioglitazone.

17. The formulation or composition according to claim 5, wherein:
the said one or more active substances is linagliptin and, optionally, another active substance selected from the group consisting of metformin, metformin HCl, pioglitazone, and pioglitazone HCl,
the first adjuvant is L-arginine, and
the optionally one or more other adjuvants is selected from the group consisting of fillers, a binder, a lubricant, a release agent, and a flow agent.

18. The formulation or composition according to claim 5, wherein the said formulation or composition comprises:
linagliptin,
metformin HCl,
L-arginine,
a binder which is copovidone,
one or more fillers selected from the group consisting of maize starch, pre-gelatinised starch and mannitol,
optionally a lubricant which is magnesium stearate, and
optionally a flow agent which is anhydrous colloidal silicon dioxide.

19. A method of preparing a formulation, composition or tablet consisting essentially of one or more active substances and L-arginine as a first adjuvant, the method comprising:
(a) preparing a combination of one or more active substances and/or L-arginine, and optionally one or more additional adjuvants into a tabletting material or mixture; and
(b) forming a formulation, composition or tablet from the tabletting material or mixture, wherein
(i) the one or more active substances and/or L-arginine undergoes systematic moisture adjusting prior to the preparation of the combination in step (a),
(ii) the tabletting material or mixture undergoes systematic moisture adjusting prior to forming the tablet in step (b); and/or
(iiI) the formulation, composition or tablet formed in step (b) undergoes systematic moisture adjusting.

20. The method according to claim 19, wherein the L-arginine is in a hydrate form.

21. The method according to claim 20, wherein the L-arginine in hydrate form contains at least about 1.5 mol water of crystallisation/1 mol L-arginine.

22. The method according to claim 19, wherein said moisture adjusting is either moisture conditioning or drying.

23. The method according to claim 19, wherein the L-arginine is selectively moisture-conditioned by:
open storage or storage of the L-arginine in a moisture-permeable package in a specific climate, or
mixing highly moisture-laden or moisture-saturated L-arginine with one or more dry adjuvants and/or active substances, or
mixing one or more moisture-laden adjuvants and/or active substances with dry L-arginine.
